(19) United States
(12) Patent Application Publication
(10) Pub. No.: US 2026/0327575 A1
(43) Pub. Date: Oct. 22, 2026

(54) PLASMID SYSTEM FOR PACKAGING RECOMBINANT ADENO-ASSOCIATED VIRUS, AND USE THEREOF

(71) Applicant: PACKGENE BIOTECH CO., LTD., Guangzhou (CN)

(72) Inventors: Huapeng Li, Guangzhou (CN); Jingluan Han, Guangzhou (CN); Ye Bu, Guangzhou (CN); Huan Chen, Guangzhou (CN); Zhiyong Dai, Guangzhou (CN)

(21) Appl. No.: 19/696,355

(22) Filed: Jun. 3, 2026

Related U.S. Application Data
(63) Continuation-in-part of application No. PCT/CN2024/130732, filed on Nov. 8, 2024.

(30) Foreign Application Priority Data
Dec. 4, 2023 (CN) 202311646675.9

(51) Int. Cl. C12N 7/00; C12N 5/071; C12N 9/12; C12N 15/86

(52) U.S. Cl. CPC C12N 7/00; C12N 5/0686; C12N 9/1241; C12N 15/86; C12N 2750/14143; C12N 2750/14152; C12N 2800/40; C12Y 207/07

(57) ABSTRACT

Provided are a plasmid system for packaging a recombinant adeno-associated virus (rAAV), and a use thereof. The plasmid system for packaging the rAAV includes: a plasmid including two inverted terminal repeats (ITRs), an adenovirus helper plasmid, and an rAAV packaging plasmid. A recognition site sequence for a recombinase and/or a coding sequence for the recombinase is inserted into at least one of the plasmid including the two ITRs, the adenovirus helper plasmid, and the rAAV packaging plasmid. During rAAV production, the plasmid system can effectively reduce the proportion of non-rAAV-related sequences erroneously packaged into the AAV capsid to improve the purity of an rAAV product, thereby greatly enhancing the druggability of the rAAV product.

### CROSS-REFERENCE TO RELATED APPLICATIONS

The present application is a Continuation-in-part Application of PCT application No. PCT/CN2024/130732 filed on Nov. 8, 2024, which claims the benefit of Chinese Patent Application No. 202311646675.9 filed on Dec. 4, 2023. The contents of the above-identified applications are hereby incorporated by reference in their entirety.

### REFERENCE TO SEQUENCE LISTING

This application includes a Sequence Listing filed electronically as an XML file named “U.S. Pat. No. 2,601,076H-PCT_SL.xml”, created on May 6, 2026, with a size of 19,769 bytes. The Sequence Listing is incorporated herein by reference.

### TECHNICAL FIELD

The present application relates to the field of biotechnologies, and specifically relates to a plasmid system for packaging a recombinant adeno-associated virus (rAAV), and a use thereof.

### BACKGROUND

rAAV, as a gene therapy vector, is characterized by high safety and strong transduction efficiency. Several rAAV-based gene therapy drugs have been approved for marketing, and many clinical trials on rAAV-based gene therapy are underway. rAAV production systems primarily include a plasmid transfection-based production system based on HEK293 cells, a production system based on insect cells and baculovirus, and a production system based on HEK293 cells and adenovirus. The plasmid transfection-based production system based on HEK293 cells enables both adherent culture and suspension culture, and can be implemented through single-plasmid or multi-plasmid transfection, which is simple and rapid. Thus, the plasmid transfection-based production system based on HEK293 cells is extensively used for rAAV production. For example, the classic triple-plasmid transfection-based production system involves co-transfection of HEK293 cells with three plasmids, including: a helper plasmid pHelper; a helper plasmid pRep-Cap; and a plasmid including a gene of interest (GOI), which may be referred to as pGOI. The helper plasmid pHelper provides nucleic acid sequences capable of expressing adenovirus helper proteins. The helper plasmid pRep-Cap provides nucleic acid sequences capable of expressing Rep and Cap proteins of AAV. Rep proteins are responsible for replication of the AAV genome and assistance in assembly of AAV particles. Cap proteins assemble into the capsid of AAV In the pGOI, the 5′ inverted terminal repeat (ITR) and the 3′ ITR in the natural genome of AAV are located upstream and downstream of the GOI, respectively.

During rAAV production, nucleic acid sequences from a plasmid backbone (such as an on nucleic acid sequence related to plasmid replication and a nucleic acid sequence of a resistance gene related to plasmid selection) can be erroneously packaged into the capsid of AAV either through an ITR/ITR-like mediated mechanism or in a random manner. This reduces the purity and potency of rAAV, causes unpredictable side effects, and poses risks to in vivo gene therapy.

### SUMMARY

An objective of the present application is to overcome the deficiencies of the prior art, and provide a plasmid system for packaging rAAV, and a use thereof.

In order to achieve the above objective, the present application adopts the following technical solutions:

In a first aspect, the present application provides a plasmid system for packaging rAAV, including: a plasmid including two ITRs, an adenovirus helper plasmid, and an rAAV packaging plasmid, where a recognition site sequence for a recombinase and/or a coding sequence for the recombinase is inserted into at least one of the plasmid including the two ITRs, the adenovirus helper plasmid, and the rAAV packaging plasmid.

The plasmid system of the present application employs a recombinase system. As a result, during rAAV production, the plasmid system can effectively reduce the proportion of non-rAAV-related sequences erroneously packaged into the AAV capsid, reduce residual nucleic acid sequences derived from a plasmid backbone in rAAV, and improve the purity of an rAAV product, thereby greatly enhancing druggability of the rAAV product.

In some such plasmid systems, the recombinase includes a tyrosine recombinase or a serine recombinase. Optionally, the tyrosine recombinase includes at least one recombinase in any system selected from the group consisting of Cre-lox, FLP-FRT, Dre-rox, and R-RS (such as cyclization recombinase (Cre recombinase), flippase recombinase, Drosophila recombinase, and R recombinase); and the serine recombinase includes at least one recombinase in any system selected from the group consisting of CinH-RS2, ParA-MRS, β-six, γδ-res, phiC31, TP901-1, R4, and Bxb1 (such as Cin recombinase, Paritioning system A resolvase, Beta recombinase, Gamma-delta resolvase, phiC31 integrase, TP901-1 integrase, R4 integrase, and Bxb1 integrase).

Site-specific recombinases can specifically recognize specific sites and mediate recombination between the specific sites, thereby achieving mutations including gene knockout, insertion, inversion, and translocation. The Cre recombinase is a recombinase derived from phage P1. The catalytic activity at the C-terminus of the Cre recombinase enables the catalytic recombination of a DNA molecule including a loxP (locus of X-over in P1) site. The loxP site includes two 13 bp palindromic sequences and an 8 bp spacer sequence. The reverse palindromic sequences serve as recognition and binding sites for the Cre recombinase. Two loxP site sequences in a same orientation are added to a circular plasmid backbone in a region close to a DNA fragment of interest, such that, upon the recognition of the loxP site sequences by the Cre recombinase, plasmid recombination occurs to produce two small circular plasmids. In this way, the DNA fragment of interest is separated from a vector backbone (VB), thereby reducing the probability of ITR/ITR-like mediated packaging of the VB.

Some such plasmid systems include: a pRep-Cap plasmid, a pHelper plasmid, and a pGOI plasmid with the recognition site sequence for the recombinase inserted; or a pRep-Cap plasmid with the recognition site sequence for the recombinase inserted, a pHelper plasmid with the recognition site sequence for the recombinase inserted, and a pGOI plasmid with the recognition site sequence for the recombinase inserted. Optionally, the plasmid system includes a GOI plasmid with loxP inserted, a pRep-Cap plasmid, and a pHelper plasmid. Optionally, the recombinase is Cre recombinase, and the recognition site sequence for the recombinase includes loxP set forth in SEQ ID NO: 2 or is set forth in SEQ ID NO: 2.

Some such plasmid systems include: a pRep-Cap plasmid, a pHelper plasmid, a pGOI plasmid with the recognition site sequence for the recombinase inserted, and a pAAVS1 plasmid with the coding sequence for the recombinase inserted; or a pRep-Cap plasmid with the recognition site sequence for the recombinase inserted, a pHelper plasmid with the recognition site sequence for the recombinase inserted, a pGOI plasmid with the recognition site sequence for the recombinase inserted, and a pAAVS1 plasmid with the coding sequence for the recombinase inserted. Optionally, the recognition site sequence for the recombinase includes a sequence set forth in SEQ ID NO: 2 (or the recognition site sequence for the recombinase is set forth in SEQ ID NO: 2), and the recombinase is Cre recombinase. Optionally, the plasmid system includes a pGOI plasmid with loxP inserted, a pRep-Cap plasmid, a pHelper plasmid, and a pAAVS1 plasmid (SEQ ID NO: 1) with a coding sequence for the Cre recombinase (GenBank accession: AY056050) inserted.

Some such plasmid systems include: a pRep-Cap plasmid, a pGOI plasmid with the recognition site sequence for the recombinase inserted, and a pHelper plasmid with the coding sequence for the recombinase inserted; or a pRep-Cap plasmid with the recognition site sequence for the recombinase inserted, a pGOI plasmid with the recognition site sequence for the recombinase inserted, and a pHelper plasmid with the coding sequence for the recombinase inserted. Optionally, the recognition site sequence for the recombinase includes a sequence set forth in SEQ ID NO: 2 (or the recognition site sequence for the recombinase is set forth in SEQ ID NO: 2), and the recombinase is Cre recombinase. Optionally, the plasmid system includes a pGOI plasmid with loxP inserted, a pRep-Cap plasmid, and a pHelper plasmid with a coding sequence for the Cre recombinase inserted.

Some such plasmid systems include any one of the following: i. a pGOI plasmid including two inverted terminal repeats (ITRs) with a loxP sequence inserted, an adenovirus helper plasmid with a sequence of Cre enzyme inserted, and an rAAV packaging plasmid; ii. a pGOI plasmid including two inverted terminal repeats (ITRs) with a loxP sequence inserted, an adenovirus helper plasmid, an rAAV packaging plasmid, and a pAAVS1 plasmid with the sequence of Cre enzyme inserted, where a 5′ terminus of a 5′ ITR and a 3′ terminus of a 3′ ITR of the pGOI plasmid are respectively added with the loxP sequence, and a nucleotide sequence of the pAAVS1 plasmid is set forth in SEQ ID NO: 1.

Some such plasmid systems include a pGOI with a loxP sequence inserted, pRep-Cap, pHelper, and a pAAVS1 with a sequence of Cre enzyme inserted. Some such plasmid systems include a pGOI with a loxP sequence inserted, pRep-Cap, and a pHelper with a sequence of Cre enzyme inserted. In some such plasmid systems, the sequence of Cre enzyme is a coding sequence for the Cre recombinase, e.g., a sequence of GenBank accession: AY056050.

In some such plasmid systems, the pGOI plasmid includes ITR sequences derived from different serotypes or optimized variants thereof. In some such plasmid systems, the pRep-Cap plasmid includes Rep protein-expressing nucleic acid sequences and Cap protein-expressing nucleic acid sequences that are derived from different serotypes, and optimized variants thereof. In some such plasmid systems, the pHelper plasmid includes adenovirus helper protein-expressing nucleic acid sequences derived from different helper virus plasmids, and optimized variants thereof.

In a second aspect, the present application provides a cell for rAAV production, where the cell stably expresses a recombinase and the cell includes the plasmid system. In some such cells, the plasmid system includes: a pRep-Cap plasmid, a pHelper plasmid, and a pGOI plasmid with the recognition site sequence for the recombinase inserted; or a pRep-Cap plasmid with the recognition site sequence for the recombinase inserted, a pHelper plasmid with the recognition site sequence for the recombinase inserted, and a pGOI plasmid with the recognition site sequence for the recombinase inserted.

Optionally, the recombinase is Cre recombinase, and the plasmid system includes a pGOI plasmid with loxP (a recognition site for the Cre recombinase) inserted, a pRep-Cap plasmid, and a pHelper plasmid.

In a third aspect, the present application provides a method for reducing residual nucleic acid sequences derived from a plasmid backbone in rAAV, including: transfecting a packaging cell with the plasmid system.

In some such methods, the packaging cell includes at least one selected from the group consisting of HEK293, HEK293T, HEK293F, HEK293A, Hela, Vero, and CHO. Optionally, the packaging cell further includes other cells for rAAV production.

In a fourth aspect, the present application provides a use of the plasmid system or the cell in rAAV production.

In a fifth aspect, the present application provides a method for producing rAAV, including: transfecting a host cell with the plasmid system; and subjecting a transfected host cell to adherent culture or suspension culture. Optionally, the host cell is selected from the group consisting of HEK293, HEK293T, HEK293F, HEK293A, Hela, Vero, and CHO. Optionally, the present application provides a method for producing rAAV, including: subjecting the cell to adherent culture or suspension culture.

Compared with the prior art, the present application includes the following beneficial effects:

The plasmid system of the present application employs a recombinase system. As a result, during rAAV production, the proportion of nucleic acid sequences derived from a plasmid backbone (such as an on nucleic acid sequence related to plasmid replication and a nucleic acid sequence of a resistance gene related to plasmid selection) erroneously packaged into the AAV capsid is greatly reduced. This can reduce residual nucleic acid sequences derived from a plasmid backbone in rAAV and effectively improve the purity of an rAAV product, thereby significantly enhancing the druggability of the rAAV product.

### BRIEF DESCRIPTION OF THE DRAWINGS

FIG. 1A, FIG. 1B, FIG. 1C, FIG. 1D, FIG. 1E, and FIG. 1F show key parameters of rAAV produced by packaging rAAV after digesting a plasmid with Cre recombinase added in vitro. A pGOI-loxP plasmid was digested by Cre recombinase added in vitro. The digested pGOI-loxP plasmid, a pRep-Cap plasmid, and a pHelper plasmid were used for rAAV production (+Cre group); and three plasmids pGOI, pRep-Cap, and pHelper were used for rAAV production (CT group). FIG. 1A, FIG. 1B, and FIG. 1C show woodchuck hepatitis virus post-transcriptional regulatory element (WPRE) titers, on titers, and reverse packaging rates of packaged rAAV, respectively; and FIG. 1D, FIG. 1E, and FIG. 1F show fold changes in WPRE titer, on titer, and reverse packaging rate of rAAV in the +Cre group compared to rAAV in the CT group, respectively.

FIG. 2 shows a map of a pAAVS1 plasmid.

FIG. 3A, FIG. 3B, FIG. 3C, FIG. 3D, FIG. 3E, and FIG. 3F show key parameters of rAAV obtained by adding a pAAVS1/Cre plasmid (i.e., a plasmid pAAVS1 expressing Cre recombinase) for rAAV packaging. Four plasmids pGOI-loxP, pRep-Cap, pHelper, and pAAVS1/Cre were used for rAAV production (+pCre group). Four plasmids pGOI-loxP, pRep-Cap, pHelper, and pAAVS1 were used for rAAV production (EV group). FIG. 3A, FIG. 3B, and FIG. 3C show WPRE titers, on titers, and reverse packaging rates of packaged rAAV, respectively; and FIG. 3D, FIG. 3E, and FIG. 3F show fold changes in WPRE titer, on titer, and reverse packaging rate of rAAV in the +pCre group compared to rAAV in the EV group, respectively.

FIG. 4A, FIG. 4B, FIG. 4C, FIG. 4D, FIG. 4E, and FIG. 4F shows key parameters of rAAV obtained by packaging rAAV after adding a nucleic acid sequence of a Cre expression cassette to a pHelper plasmid. Three plasmids pGOI-loxP, pRep-Cap, and pHelper/Cre were used for rAAV production (+Cre group). Three plasmids pGOI-loxP, pRep-Cap, and pHelper were used for rAAV production (EV group). FIG. 4A, FIG. 4B, and FIG. 4C show WPRE titers, on titers, and reverse packaging rates of packaged rAAV, respectively; and FIG. 4D, FIG. 4E, and FIG. 4F show fold changes in WPRE titer, on titer, and reverse packaging rate of rAAV in the +Cre group compared to rAAV in the EV group, respectively.

FIG. 5A, FIG. 5B, FIG. 5C, FIG. 5D, FIG. 5E, and FIG. 5F shows key parameters of rAAV produced through rAAV packaging using a cell Cre-KI stably expressing the Cre recombinase. Three plasmids pGOI-loxP, pRep-Cap, and pHelper were transfected into the cell Cre-KI stably expressing the Cre recombinase for rAAV production (Cre-KI group). Three plasmids pGOI, pRep-Cap, and pHelper were transfected into a cell 293T for rAAV production (293T group). FIG. 5A, FIG. 5B, and FIG. 5C show WPRE titers, on titers, and reverse packaging rates of packaged rAAV, respectively; and FIG. 5D, FIG. 5E, and FIG. 5F show fold changes in WPRE titer, on titer, and reverse packaging rate of rAAV in the Cre-KI group compared to rAAV in the 293T group, respectively.

### DETAILED DESCRIPTION

To well explain the objective, technical solutions, and advantages of the present application, the present application will be further explained below with reference to specific examples. It should be understood by those skilled in the art that the specific examples described herein are merely intended to explain the present application, rather than to limit the present application.

In the following examples, unless otherwise specified, the experimental methods adopted are conventional, and the materials and reagents adopted are commercially available. The WPRE titer refers to a copy number of a DNA fragment of interest. The on titer refers to a copy number of VB. The reverse packaging rate refers to a ratio of a copy number of VB to a copy number of a DNA fragment of interest.

The term “ITR” refers to nucleotide sequences that are oppositely oriented, complementary to each other, and located at two termini of a DNA element (such as transposon, viral genome, or specific gene cassette). For example, ITR on the AAV genome is a DNA sequence with a length of approximately 145 base pairs (bp), and is the only cis-acting element required for viral replication and packaging. In some examples, a specific nucleotide sequence of the “ITR” is ttggccactccctctctgcgcgctcgctcgctcactgaggccgggcgaccaaaggtcgcccgacgcccgggctttgcccgggcggcctc agtgagcgagcgagcgcgcagagagggagtggccaactccatcactaggggttcct (SEQ ID NO: 15).

The term “packaging cell” refers to a specialized cell line that has been selected, modified, and optimized to enable large-scale, stable, and efficient production of a viral vector. The packaging cell plays an important role in fields such as gene therapy, gene function research, and vaccine development. The packaging cell typically harbors a helper gene, is amenable to engineering manipulation, and is suitable for industrial production.

### Example 1: RAAV Packaging Using a Plasmid Digested with Cre Recombinase Added In Vitro

### (1) Vector Construction and Plasmid Extraction

Based on sequence information of a Cre recognition site loxP (ATAACTTCGTATAGCATACATTATACGAAGTTAT, SEQ ID NO: 2) and information of a pGOI plasmid (ADDGENE: 27970), a pair of chimeric primers including the loxP sequence (loxP-VB-F: tcacaccgcatacgtcaaagcaaccATAACTTCGTATAGCATACATTATACGAAGTTATATagtacgcgccct gtagcg, SEQ ID NO: 3; and loxP-VB-R: gaaccgtaaaaaggccgcgttgctgATAACTTCGTATAATGTATGCTATACGAAGTTATgcgtttttccataggc tccg, SEQ ID NO: 4) were designed for amplifying VB, and primers (GOI-F: cagcaacgcggcctttttacggttc, SEQ ID NO: 5; and GOI-R: ggttgctttgacgtatgcggtgtga, SEQ ID NO: 6) for amplifying a GOI fragment were designed.

Then, the pGOI plasmid was amplified using loxP-VB-F/loxP-VB-R and GOI-F/GOI-R separately. An amplification system was as follows: 25 μL of 2×KOD OnePCR Master Mix (TOYOBO, #KMM-101), 1.0 μL of a forward primer (10 μM), 1.0 μL of a reverse primer (10 μM), 20 ng of a DNA template, and the balance of sterilized water, with a final volume of 50 μL. Amplification conditions were as follows: pre-denaturation at 95 °C for 3 min; denaturation at 98 °C for 10 s, annealing at 58 °C for 5 s, and extension at 68 °C for 10 sec/kb (32 cycles); and extension at 68 °C for 3 min. After amplification, 1.0% (w/v) agarose gel electrophoresis was conducted. Gel slices including DNA fragments of interest were excised. DNAs of interest were recovered and purified using HiPure Gel Pure DNA Micro Kits (MEGAN, #D21111-03) to obtain the GOI fragment and the VB fragment.

The two DNA fragments obtained after recovery and purification were ligated by the Gibson Assembly method. A ligation system was as follows: 15 μL of ¾× Gibson Assembly Master Mix, 100 ng of the VB fragment, 100 ng of the GOI fragment, and the balance of sterilized water, with a final volume of 20 μL. The ligation system was thoroughly mixed and then incubated at 50 °C for 30 min. A ligated DNA product was transformed into XL10-Gold Chemically Competent Cells (Shanghai Weidi Biotechnology Co., Ltd., #DL1050). Screening was conducted using an ampicillin-containing LB medium. Single clones were then picked and sent to Beijing Tsingke Biotech Co., Ltd. for sequencing. A plasmid in which a loxP sequence was added to each of a 5′ terminus of a 5′ ITR and a 3′ terminus of a 3′ ITR of the pGOI plasmid was finally obtained, which was named pGOI-loxP.

High-concentration and high-purity plasmids pGOI, pGOI-loxP, pRep-Cap (ADDGENE: 112865), and pHelper (GenBank: AF369965) were extracted. The pGOI-loxP plasmid was recombined in vitro with Cre recombinase (NEW ENGLAND BIOLABS, #M0298S) added. A recombination reaction system was as follows: 5.0 μL of 10× Cre buffer, 4.0 μL of the Cre recombinase, 1.0 μg of the pGOI-loxP plasmid, and the balance of sterilized water, with a final volume of 50 μL. Recombination reaction conditions were as follows: incubation at 37 °C for 50 min and incubation at 70 °C for 10 min. After a recombination reaction was completed, a recombination product was purified using HiPure Gel Pure DNA Micro Kit.

(2) rAAV Packaging and Titer Determination

One day before transfection, 293T cells were resuspended in Dulbecco's Modified Eagle Medium (DMEM), counted, uniformly spread into 10-cm cell culture dishes at a density of 6×10⁶ cells/dish, and then cultured at 37 °C and 5% (v/v) CO₂ until a cell confluency reached approximately 80%.

The following groups were set: a CT group: pGOI, pRep-Cap, and pHelper; and a +Cre group: pGOI-loxP recombined with the Cre recombinase, pRep-Cap, and pHelper.

On the day of transfection, an original medium was replaced with fresh DMEM. pGOI, pRep-Cap, and pHelper (CT group) or pGOI-loxP recombined with the Cre recombinase, pRep-Cap, and pHelper (+Cre group) were mixed at a ratio of 2.0 μg:2.5 μg:2.5 g. Three replicates were set for each group. 0.5 mL of DMEM was mixed with plasmid DNA to produce a DNA solution. 0.5 mL of DMEM was mixed with 8.4 μL of PEIpro to produce a PEIpro solution. Then, the PEIpro solution and the DNA solution were mixed thoroughly and allowed to stand at room temperature for 15 min to produce a DNA-PEIpro mixed solution. The DNA-PEIpro mixed solution was gently mixed with well-grown cells, and the cells were further cultured in an incubator.

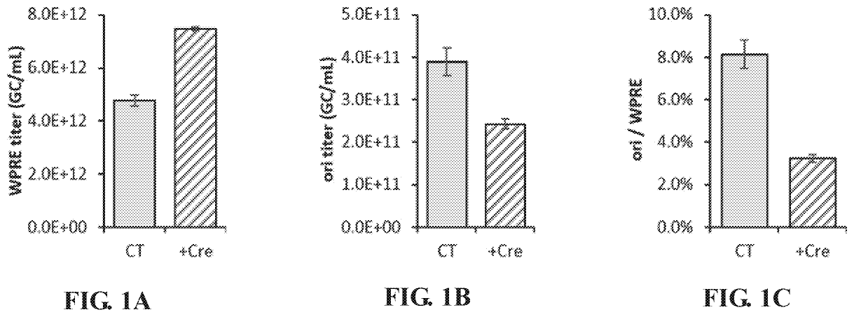

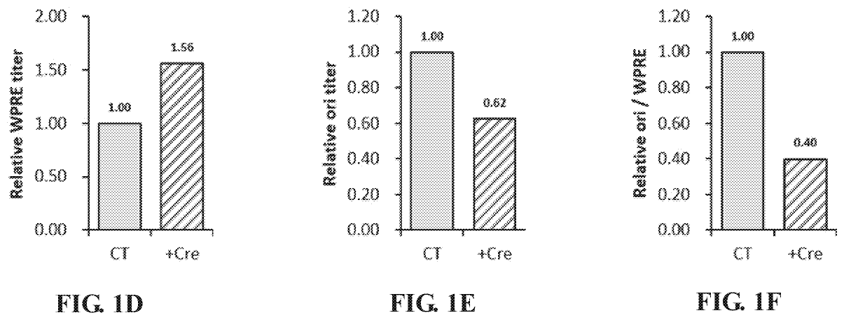

At 72 h after transfection, cells were lysed with 1% (v/v) chloroform, and a lysate was then collected into a 50 mL centrifuge tube. A nuclease was added for digestion. rAAV was concentrated and purified with PEG8000. Purified rAAV was digested with DNase I. A WPRE titer and an on titer were determined by quantitative reverse transcription polymerase chain reaction (qRT-PCR). A standard curve was plotted using serially diluted plasmid standards. A WPRE titer and an ori titer of an rAAV sample were calculated based on the standard curve. Results are shown in FIG. 1A, FIG. 1B, FIG. 1D, and FIG. 1E.

A pGOI-loxP plasmid (digested in vitro with Cre recombinase), the pRep-Cap plasmid, and the pHelper plasmid were used for rAAV production (+Cre group). A proportion of the plasmid backbone reverse-packaged into an rAAV capsid (reverse packaging rate) in rAAV produced in the +Cre group was 0.4-fold a reverse packaging rate of the CT group (control) in which rAAV was produced using a combination of the three plasmids pGOI, pRep-Cap, and pHelper (as shown in FIG. 1C and FIG. 1F).

### Example 2: RAAV Packaging with a pAAVS1/Cre Plasmid Added

### (1) Vector Construction and Plasmid Extraction

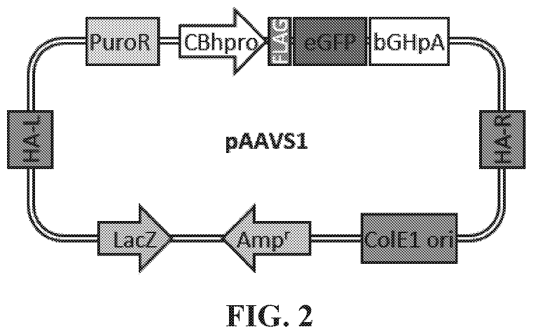

Sequence information of Cre (GenBank accession: AY056050) was acquired from the GenBank database, and accordingly, gene synthesis was conducted. Primers (Cre-F: ttacaaagacgatgacgataagATGTCCAATCTCCTGACTGTTCA, SEQ ID NO: 7; and Cre-R: agcgagctctaggaattcttaTCAGTCACCATCTTCGAGCAGTC, SEQ ID NO: 8) for amplifying a Cre fragment and primers (VB-F: tgataagaattcctagagctcgct, SEQ ID NO: 9; and VB-R: catcttatcgtcatcgtctttgtaa, SEQ ID NO: 10) for amplifying a backbone of a pAAVS1 plasmid (a map of the plasmid is shown in FIG. 2, and a nucleotide sequence of the plasmid is set forth in SEQ ID NO: 1) were designed.

The Cre fragment was amplified using Cre-F/Cre-R, and the backbone of the pAAVS1 plasmid was amplified using VB-F/VB-R. An amplification system was as follows: 25 μL of 2×KOD OnePCR Master Mix, 1.0 μL of a forward primer (10 μM), 1.0 μL of a reverse primer (10 μM), 20 ng of a DNA template, and the balance of sterilized water, with a final volume of 50 μL. Amplification conditions were as follows: pre-denaturation at 95 °C for 3 min; denaturation at 98 °C for 10 s, annealing at 58 °C for 5 s, and extension at 68 °C for 10 sec/kb (32 cycles); and extension at 68 °C for 3 min. After amplification, 1.0% (w/v) agarose gel electrophoresis was conducted. Gel slices including DNA fragments of interest were excised. DNAs of interest were recovered and purified using HiPure Gel Pure DNA Micro Kits to obtain the Cre gene fragment and the pAAVS1-VB fragment.

The two DNA fragments obtained after recovery and purification were ligated by the Gibson Assembly method. A ligation system was as follows: 7.5 μL of ¾× Gibson Assembly Master Mix, 100 ng of the Cre gene fragment, 100 ng of the pAAVS1-VB fragment, and the balance of sterilized water, with a final volume of 10 μL. The ligation system was thoroughly mixed and then incubated at 50 °C for 30 min. A ligated DNA product was transformed into XL10-Gold Chemically Competent Cells. Screening was conducted using an ampicillin-containing LB medium. Single clones were then picked and sent to Beijing Tsingke Biotech Co., Ltd. for sequencing. A plasmid in which a Cre coding frame sequence (namely, a coding region sequence) was inserted into the pAAVS1 plasmid was finally obtained, which was named pAAVS1/Cre.

High-concentration and high-purity plasmids pGOI-loxP, pRep-Cap, pHelper, pAAVS1, and pAAVS1/Cre were extracted.

(2) rAAV Packaging and Titer Determination

One day before transfection, 293T cells were resuspended in DMEM, counted, uniformly spread into 10-cm cell culture dishes at a density of 6×10⁶ cells/dish, and then cultured at 37 °C and 5% (v/v) CO₂ until a cell confluency reached approximately 80%.

The following groups were set: an EV group: pGOI-loxP, pRep-Cap, pHelper, and pAAVS1; and a +Cre group: pGOI-loxP, pRep-Cap, pHelper, and pAAVS1/Cre.

On the day of transfection, an original medium was replaced with fresh DMEM. pGOI-loxP, pRep-Cap, pHelper, and pAAVS1 (EV group) or pGOI-loxP, pRep-Cap, pHelper, and pAAVS1/Cre (+Cre group) were mixed at a ratio of 2.0 μg:2.5 μg:2.5 μg:0.5 g. Three replicates were set for each group. 0.5 mL of DMEM was mixed with plasmid DNA to produce a DNA solution. 0.5 mL of DMEM was mixed with 9.0 μL of PEIpro to produce a PEIpro solution. Then, the PEIpro solution and the DNA solution were mixed thoroughly and allowed to stand at room temperature for 15 min to produce a DNA-PEIpro mixed solution. The DNA-PEIpro mixed solution was gently mixed with well-grown cells, and the cells were further cultured in an incubator.

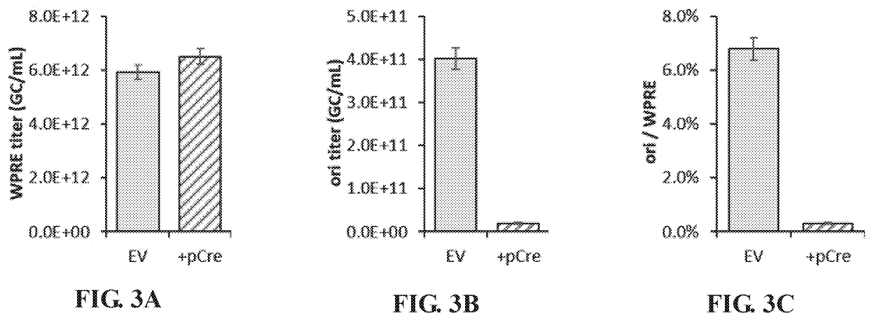

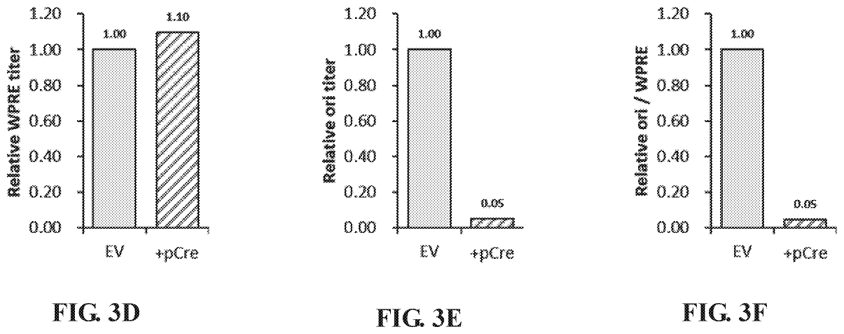

At 72 h after transfection, cells were lysed with 1% (v/v) chloroform, and a lysate was then collected into a 50 mL centrifuge tube. A nuclease was added for digestion. rAAV was concentrated and purified with PEG8000. Purified rAAV was digested with DNase I. A WPRE titer and an on titer were determined by qRT-PCR. A standard curve was plotted using serially diluted plasmid standards. A WPRE titer and an ori titer of an rAAV sample were calculated based on the standard curve. Results are shown in FIG. 3A, FIG. 3B, FIG. 3D, and FIG. 3E.

A combination of the four plasmids pGOI-loxP, pRep-Cap, pHelper, and pAAVS1/Cre was used for rAAV production (+Cre group). A proportion of the plasmid backbone reverse-packaged into an rAAV capsid (reverse packaging rate) in rAAV produced in the +Cre group was 0.05-fold a reverse packaging rate of the EV group (control) in which rAAV was produced using a combination of the four plasmids pGOI-loxP, pRep-Cap, pHelper, and pAAVS1 (as shown in FIG. 3C and FIG. 3F).

### Example 3: rAAV Packaging with a pHelper Plasmid Including a Nucleic Acid Sequence of a Cre Expression Cassette

### (1) Vector Construction and Plasmid Extraction

Primers for amplifying a Cre expression cassette-containing fragment (pHel-Cre-F: acgtcgacgtttaaaccaCGTTACATAACTTACGGTAAATGG, SEQ ID NO: 11; and pHel-Cre-R: gcctttgagtgagctgatcatatgTCCCCAGCATGCCTGCTATTCTC, SEQ ID NO: 12) were designed.

pAAVS1/Cre was amplified using pHel-Cre-F/pHel-Cre-R. An amplification system was as follows: 25 μL of 2×KOD OnePCR Master Mix, 1.0 μL of a forward primer (10 μM), 1.0 μL of a reverse primer (10 μM), 20 ng of a DNA template, and the balance of sterilized water, with a final volume of 50 μL. Amplification conditions were as follows: pre-denaturation at 95 °C for 3 min; denaturation at 98 °C for 10 s, annealing at 58 °C for 5 s, and extension at 68 °C for 10 sec/kb (32 cycles); and extension at 68 °C for 3 min. After amplification, 1.0% (w/v) agarose gel electrophoresis was conducted. A gel slice including a DNA fragment of interest was excised. DNA of interest was recovered and purified using HiPure Gel Pure DNA Micro Kit to obtain a Cre expression cassette fragment.

A pHelper plasmid (GenBank: AF369965) was digested using NdeI (THERMOFISHER SCIENTIFIC, #FD0583). A digestion system was as follows: 5.0 μL of 10× FastDigest buffer, 2.0 μL of NdeI, 2.0 μg of the pHelper plasmid, and the balance of sterilized water, with a final volume of 50 μL. Digestion conditions were as follows: incubation at 37 °C for 30 min and incubation at 65 °C for 5 min. After digestion, a product was purified using HiPure Gel Pure DNA Micro Kit.

The Cre expression cassette fragment and a digested pHelper plasmid were ligated by the Gibson Assembly method. A ligation system was as follows: 15 μL of ¾× Gibson Assembly Master Mix, 20 ng of the digested pHelper plasmid, 10 ng of the Cre expression cassette fragment, and the balance of sterilized water, with a final volume of 20 μL. The ligation system was thoroughly mixed and then incubated at 50 °C for 30 min. A ligated DNA product was transformed into XL10-Gold Chemically Competent Cells. Screening was conducted using an ampicillin-containing LB medium. Single clones were then picked and sent to Beijing Tsingke Biotech Co., Ltd. for sequencing. A plasmid in which the Cre expression cassette fragment was inserted into the pHelper plasmid was finally obtained, which was named pHelper/Cre.

High-concentration and high-purity plasmids pGOI-loxP, pRep-Cap, pHelper, and pHelper/Cre were extracted.

(2) rAAV Packaging and Titer Determination

One day before transfection, 293T cells were resuspended in DMEM, counted, uniformly spread into 10-cm cell culture dishes at a density of 6×10⁶ cells/dish, and then cultured at 37 °C and 5% (v/v) CO₂ until a cell confluency reached approximately 80%.

The following groups were set: an EV group: pGOI-loxP, pRep-Cap, and pHelper; and a +Cre group: pGOI-loxP, pRep-Cap, and pHelper/Cre.

On the day of transfection, an original medium was replaced with fresh DMEM. pGOI-loxP, pRep-Cap, and pHelper (EV group) or pGOI-loxP, pRep-Cap, and pHelper/Cre (+Cre group) were mixed at a ratio of 2.0 μg:2.5 μg:2.5 g. Three replicates were set for each group. 0.5 mL of DMEM was mixed with plasmid DNA to produce a DNA solution. 0.5 mL of DMEM was mixed with 8.4 μL of PEIpro to produce a PEIpro solution. Then, the PEIpro solution and the DNA solution were mixed and allowed to stand at room temperature for 15 min to produce a DNA-PEIpro mixed solution. The DNA-PEIpro mixed solution was gently mixed with well-grown cells, and the cells were further cultured in an incubator.

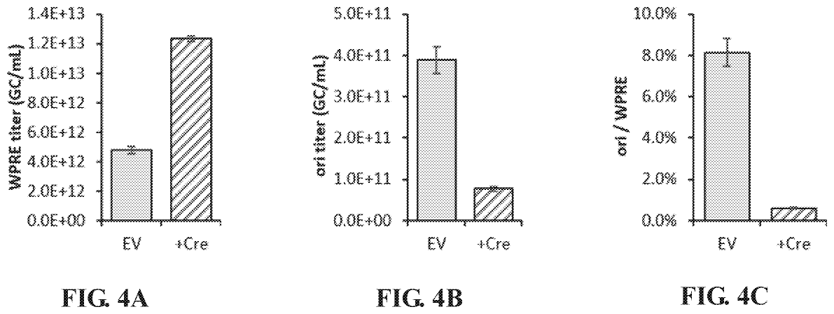

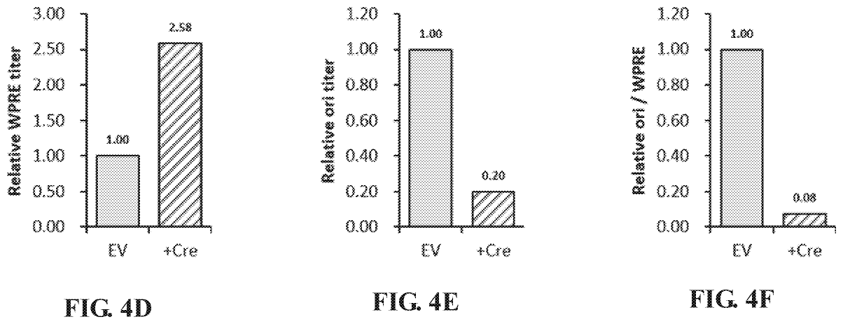

At 72 h after transfection, cells were lysed with 1% (v/v) chloroform, and a lysate was then collected into a 50 mL centrifuge tube. A nuclease was added for digestion. rAAV was concentrated and purified with PEG8000. Purified rAAV was digested with DNase I. A WPRE titer and an on titer were determined by qRT-PCR. A standard curve was plotted using serially diluted plasmid standards. A WPRE titer and an ori titer of an rAAV sample were calculated based on the standard curve. Results are shown in FIG. 4A, FIG. 4B, FIG. 4D, and FIG. 4E.

A combination of the three plasmids pGOI-loxP, pRep-Cap, and pHelper/Cre was used for rAAV production (+Cre group). A proportion of the plasmid backbone reverse-packaged into an rAAV capsid (reverse packaging rate) in rAAV produced in the +Cre group was 0.08-fold a reverse packaging rate of the EV group (control) in which rAAV was produced using a combination of the three plasmids pGOI-loxP, pRep-Cap, and pHelper (as shown in FIG. 4C and FIG. 4F).

### Example 4: RAAV Packaging Using Cre-KI Cells

### (1) Vector Construction and Plasmid Extraction

sgAAVS1 primers (sgAAVS1-F: CACCtaaggaatctgcctaacagg, SEQ ID NO: 13; and sgAAVS1-R: AACcctgttaggcagattcctta, SEQ ID NO: 14) targeting an AAVS1 site (also known as a PPP1R2C site, an open locus on human chromosome 19) were designed. A primer annealing system was as follows: 2.0 μL of a forward primer (10 μM), 2.0 μL of a reverse primer (10 μM), and the balance of sterilized water, with a final volume of 20 μL. The primer annealing system was incubated at 98 °C for 5 min and then cooled naturally to room temperature.

A pX330 plasmid (ADDGENE: 42230) was digested with BpiI (THERMOFISHER SCIENTIFIC, #FD1014). A digestion system was as follows: 5.0 μL of 10× FastDigest buffer, 2.0 μL of BpiI, 2.0 μg of the pX330 plasmid, and the balance of sterilized water, with a final volume of 50 μL. Digestion conditions were as follows: incubation at 37 °C for 30 min and incubation at 65 °C for 5 min. After digestion, a product was purified using HiPure Gel Pure DNA Micro Kit.

A DNA ligation system was as follows: 1.0 μL of 10× T4 DNA ligase buffer, 1.0 μL of T4 DNA ligase (TAKARA, #6022), 0.5 μL of a pX330 digested product, 2.0 μL of annealed primers, and the balance of sterilized water, with a final volume of 10 μL. The DNA ligation system was thoroughly mixed and then incubated at 16 °C for about 2 h. A ligated DNA product was transformed into XL10-Gold Chemically Competent Cells. Screening was conducted using an ampicillin-containing LB medium. Single clones were then picked and sent to Beijing Tsingke Biotech Co., Ltd. for sequencing. A plasmid in which the sgAAVS1 target site was inserted into the pX330 plasmid was finally obtained, which was named pX330/sgAAVS1.

High-concentration and high-purity plasmids pGOI, pGOI-loxP, pRep-Cap, pHelper, pAAVS1/Cre, and pX330/sgAAVS1 were extracted.

### (2) Cell Transfection and Screening

One day before transfection, 293T cells were resuspended in DMEM, counted, and uniformly spread into a 6-well cell culture plate at a density of 1×10⁶ cells/well. On the day of transfection, the pAAVS1/Cre plasmid and the pX330/sgAAVS1 plasmid were mixed at a ratio of 2.0 μg:1.0 g, 6.0 μL of a P3000 reagent and 125 μL of Opti-MEM I were then added, and thorough mixing was conducted to produce a DNA-P3000 mixed solution. 3.75 μL of Lipofectamine® 3000 and 125 μL of Opti-MEM I were thoroughly mixed in a centrifuge tube to produce a Lipofectamine® 3000 mixed solution. Then, the DNA-P3000 mixed solution and the Lipofectamine® 3000 mixed solution were thoroughly mixed and allowed to stand at room temperature for 15 min. A resulting transfection mixture was added to the 6-well cell culture plate, and mixing was conducted gently. The 6-well cell culture plate was then incubated at 37 °C and 5% (v/v) CO₂ for about 48 h to 72 h.

Transfected cells were collected, resuspended in DMEM, and counted. The transfected cells were inoculated into a 6-well cell culture plate at a density of 1×10⁶ cells/well. Puromycin was added to a final concentration of 1.0 μg/mL, and the transfected cells were cultured at 37 °C and 5% (v/v) CO₂ for about 1 week to 2 weeks to allow screening. Single colonies were then isolated from the 96-well cell culture plate and expanded to obtain cells designated as Cre-KI.

(3) rAAV Packaging and Titer Determination

One day before transfection, 293T and Cre-KI cells were each resuspended in DMEM, counted, uniformly spread into 10-cm cell culture dishes at a density of 6×10⁶ cells/dish, and then cultured at 37 °C and 5% (v/v) CO₂ until a cell confluency reached approximately 80%.

On the day of transfection, an original medium was replaced with fresh DMEM. pGOI, pRep-Cap, and pHelper (293T group) or pGOI-loxP, pRep-Cap, and pHelper (Cre-KI group) were mixed at a ratio of 2.0 g. 2.5 g. 2.5 g. Three replicates were set for each group. 0.5 mL of DMEM was mixed with plasmid DNA to produce a DNA solution. 0.5 mL of DMEM was mixed with 8.4 μL of PEIpro to produce a PEIpro solution. Then, the PEIpro solution and the DNA solution were mixed thoroughly and allowed to stand at room temperature for 15 min to produce a DNA-PEIpro mixed solution. The DNA-PEIpro mixed solution was gently mixed with cultured cells, and the cells were further cultured in an incubator.

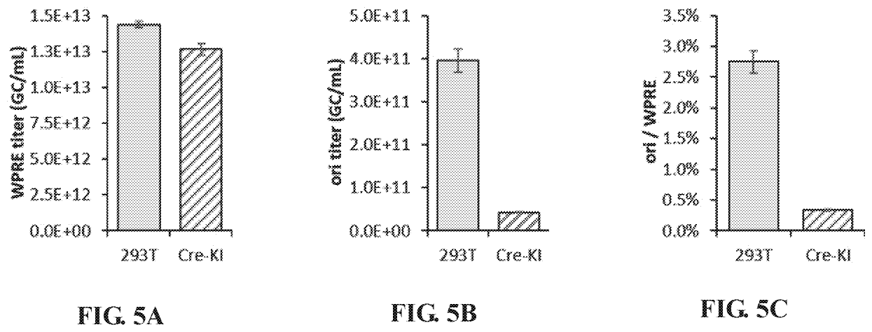

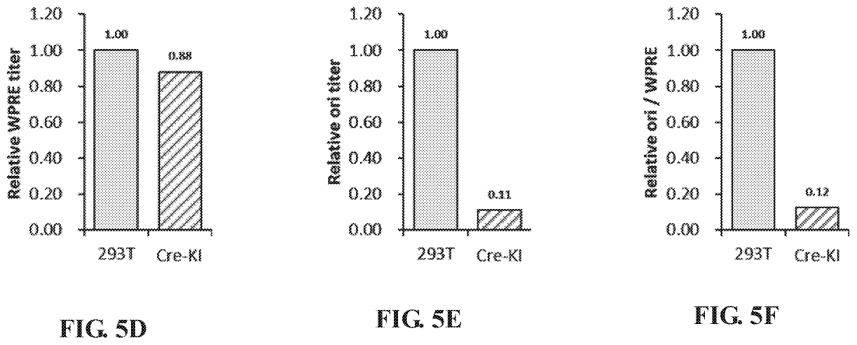

At 72 h after transfection, cells were lysed with 1% (v/v) chloroform, and a lysate was then collected into a 50 mL centrifuge tube. A nuclease was added for digestion. rAAV was concentrated and purified with PEG8000. Purified rAAV was digested with DNase I. A WPRE titer and an on titer were determined by qRT-PCR. A standard curve was plotted using serially diluted plasmid standards. A WPRE titer and an on titer of an rAAV sample were calculated based on the standard curve. Results are shown in FIG. 5A, FIG. 5B, FIG. 5D, and FIG. 5E.

The three plasmids pGOI-loxP, pRep-Cap, and pHelper were co-transfected into Cre-KI cells stably expressing Cre to enable rAAV production (Cre-KI group). The three plasmids pGOI, pRep-Cap, and pHelper were co-transfected into 293T cells to enable rAAV production (293T group). For rAAV produced in the Cre-KI group, a proportion of a plasmid backbone reverse-packaged into an rAAV capsid (reverse packaging rate) was 0.12-fold a reverse packaging rate of the 293T group (control) (as shown in FIG. 5C and FIG. 5F).

Finally, it should be noted that the above examples are provided merely to describe the technical solutions of the present application, rather than to limit the scope of protection of the present application. Although the present application is described in detail with reference to preferred examples, those of ordinary skill in the art should understand that modifications or equivalent replacements may be made to the technical solutions of the present application without departing from the spirit and scope of the technical solutions of the present application.

1. A plasmid system for packaging a recombinant adeno-associated virus (rAAV), comprising: a plasmid comprising two inverted terminal repeats (ITRs), an adenovirus helper plasmid, and an rAAV packaging plasmid,
wherein a recognition site sequence for a recombinase and/or a coding sequence for the recombinase is inserted into at least one of the plasmid comprising the two ITRs, the adenovirus helper plasmid, and the rAAV packaging plasmid.

2. The plasmid system according to claim 1, wherein the recombinase comprises a tyrosine recombinase or a serine recombinase.

3. The plasmid system according to claim 2, wherein the tyrosine recombinase comprises at least one recombinase in any system selected from the group consisting of Cre-lox, FLP-FRT, Dre-rox, and R-RS; and
the serine recombinase comprises at least one recombinase in any system selected from the group consisting of CinH-RS2, ParA-MRS, β-six, γδ-res, phiC31, TP901-1, R4, and Bxb1.

4. The plasmid system according to claim 1, wherein the plasmid system comprises: a pRep-Cap plasmid, a pHelper plasmid, and a pGOI plasmid with the recognition site sequence for the recombinase inserted; or
a pRep-Cap plasmid with the recognition site sequence for the recombinase inserted, a pHelper plasmid with the recognition site sequence for the recombinase inserted, and a pGOI plasmid with the recognition site sequence for the recombinase inserted.

5. The plasmid system according to claim 4, wherein the recombinase is cyclization recombinase (Cre recombinase), and the recognition site sequence for the recombinase comprises loxP set forth in SEQ ID NO: 2.

6. The plasmid system according to claim 1, wherein the plasmid system comprises: a pRep-Cap plasmid, a pHelper plasmid, a pGOI plasmid with the recognition site sequence for the recombinase inserted, and a pAAVS1 plasmid with the coding sequence for the recombinase inserted; or
a pRep-Cap plasmid with the recognition site sequence for the recombinase inserted, a pHelper plasmid with the recognition site sequence for the recombinase inserted, a pGOI plasmid with the recognition site sequence for the recombinase inserted, and a pAAVS1 plasmid with the coding sequence for the recombinase inserted.

7. The plasmid system according to claim 6, wherein the recognition site sequence for the recombinase comprises a sequence set forth in SEQ ID NO: 2, and the recombinase is Cre recombinase.

8. The plasmid system according to claim 1, wherein the plasmid system comprises: a pRep-Cap plasmid, a pGOI plasmid with the recognition site sequence for the recombinase inserted, and a pHelper plasmid with the coding sequence for the recombinase inserted; or
a pRep-Cap plasmid with the recognition site sequence for the recombinase inserted, a pGOI plasmid with the recognition site sequence for the recombinase inserted, and a pHelper plasmid with the coding sequence for the recombinase inserted.

9. The plasmid system according to claim 8, wherein the recognition site sequence for the recombinase comprises a sequence shown in SEQ ID NO: 2, and the recombinase is Cre recombinase.

10. A cell for rAAV production, wherein the cell stably expresses a recombinase and the cell comprises the plasmid system according to claim 4.

11. A cell for rAAV production, wherein the cell stably expresses a recombinase and the cell comprises the plasmid system according to claim 5.

12. A method for reducing residual nucleic acid sequences derived from a plasmid backbone in rAAV, comprising: transfecting a packaging cell with the plasmid system according to claim 1.

13. A method for reducing residual nucleic acid sequences derived from a plasmid backbone in rAAV, comprising: transfecting a packaging cell with the plasmid system according to claim 4.

14. A method for reducing residual nucleic acid sequences derived from a plasmid backbone in rAAV, comprising: transfecting a packaging cell with the plasmid system according to claim 6.

15. The method according to claim 12, wherein the packaging cell comprises at least one selected from the group consisting of HEK293, HEK293T, HEK293F, HEK293A, Hela, Vero, and CHO.

16. The method according to claim 13, wherein the packaging cell comprises at least one selected from the group consisting of HEK293, HEK293T, HEK293F, HEK293A, Hela, Vero, and CHO.

17. A method for producing rAAV, comprising:
transfecting a host cell with the plasmid system according to claim 1; and
subjecting a transfected host cell to adherent culture or suspension culture.

18. A method for producing rAAV, comprising:
transfecting a host cell with the plasmid system according to claim 6; and
subjecting a transfected host cell to adherent culture or suspension culture.

19. A method for producing rAAV, comprising: subjecting the cell according to claim 10 to adherent culture or suspension culture.

20. A method for producing rAAV, comprising: subjecting the cell according to claim 11 to adherent culture or suspension culture.


SEQUENCE LISTING (15 sequences)

SEQ ID NO: 1 - DNA, 5091 bp, synthetic construct
  source 1..5091: mol_type = other DNA
gtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggagggagagcttggcagggtttaagagctatgctggaaacagcatagcaagtttaaataaggctagtccgttatcaacttgaaaaagtggcaccgagtcggtgctttttttgcggccgcctcgagtgatcaaaaaaccaacacacgcttccaatgaaaataaacgatcctttattgctagcctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatcccgggcccgcggtacccagcttggaggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccatacggatctgcgatgataagctgtcaaacatgagaattggtcgacgatatcagatctgaggaacccctagtgatggagttggccactccctctctgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccactagtatgcatcctgcaggacgcgtcctacccactccctctatgcgcgctcgctcactcactcggccctggagacctttggtctccagggccgagtgagtgagcgagcgcgcatagagggagtgggtacctccatcatctggtttcccagatctgatatcggcgcgcccgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccac

SEQ ID NO: 2 - DNA, 34 bp, synthetic construct
  source 1..34: mol_type = other DNA
ataacttcgtatagcatacattatacgaagttat

SEQ ID NO: 3 - DNA, 79 bp, synthetic construct
  source 1..79: mol_type = other DNA
tcacaccgcatacgtcaaagcaaccataacttcgtatagcatacattatacgaagttatatagtacgcgccctgtagcg

SEQ ID NO: 4 - DNA, 79 bp, synthetic construct
  source 1..79: mol_type = other DNA
gaaccgtaaaaaggccgcgttgctgataacttcgtataatgtatgctatacgaagttatgcgtttttccataggctccg

SEQ ID NO: 5 - DNA, 25 bp, synthetic construct
  source 1..25: mol_type = other DNA
cagcaacgcggcctttttacggttc

SEQ ID NO: 6 - DNA, 25 bp, synthetic construct
  source 1..25: mol_type = other DNA
ggttgctttgacgtatgcggtgtga

SEQ ID NO: 7 - DNA, 45 bp, synthetic construct
  source 1..45: mol_type = other DNA
ttacaaagacgatgacgataagatgtccaatctcctgactgttca

SEQ ID NO: 8 - DNA, 44 bp, synthetic construct
  source 1..44: mol_type = other DNA
agcgagctctaggaattcttatcagtcaccatcttcgagcagtc

SEQ ID NO: 9 - DNA, 24 bp, synthetic construct
  source 1..24: mol_type = other DNA
tgataagaattcctagagctcgct

SEQ ID NO: 10 - DNA, 25 bp, synthetic construct
  source 1..25: mol_type = other DNA
catcttatcgtcatcgtctttgtaa

SEQ ID NO: 11 - DNA, 42 bp, synthetic construct
  source 1..42: mol_type = other DNA
acgtcgacgtttaaaccacgttacataacttacggtaaatgg

SEQ ID NO: 12 - DNA, 47 bp, synthetic construct
  source 1..47: mol_type = other DNA
gcctttgagtgagctgatcatatgtccccagcatgcctgctattctc

SEQ ID NO: 13 - DNA, 24 bp, synthetic construct
  source 1..24: mol_type = other DNA
cacctaaggaatctgcctaacagg

SEQ ID NO: 14 - DNA, 23 bp, synthetic construct
  source 1..23: mol_type = other DNA
aaccctgttaggcagattcctta

SEQ ID NO: 15 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct